(19) United States
(12) Patent Application Publication
(10) Pub. No.: US 2004/0110696 A1
(43) Pub. Date: Jun. 10, 2004

(54) GENE THERAPY SYSTEM AND METHOD USING ALPHA-MSH AND ITS DERIVATIVES

(76) Inventors: James M Lipton, Woodland Hills, CA (US); Anna P Catania, Milan (IT)

Correspondence Address: PERKINS COIE LLP, POST OFFICE BOX 1208, SEATTLE, WA, 98111-1208, US

(21) Appl. No.: 10/258,976

(22) PCT Filed: Apr. 27, 2001
(86) PCT No.: PCT/US01/13826

(51) Int. Cl. A61K 48/00

(52) U.S. Cl. 514/44; 435/320.1

(57) ABSTRACT

Disclosed in this specification is the use alpha-MSH and/or its derivatives as an adjunct to gene therapy. In one aspect, the gene therapy vector includes nucleic acids that expresses alpha-MSH and/or its derivatives. Inflammatory or immune response gene promoter may control the expression of alpha-MSH and/or its derivatives. The sequences may also be expressed together with a therapeutic gene using an internal ribosomal entry site sequence. In another aspect, pharmacologically effective amount of alpha-MSH and/or its derivatives may be administered before, after, or concurrently with the gene therapy vector carrying the appropriate gene.

### FIELD OF THE INVENTION

The present invention relates to the field of gene therapy.

### BACKGROUND OF THE INVENTION

Various diseases originate from defective genes that are either inherited or modified during life by environmental agents. Examples of these diseases include different forms of cancer, hemophilia, or LDL receptor deficiency. Gene therapy or gene replacement therapy promises a fundamental cure for these diseases by replacing, augmenting, or inhibiting these defective genes.

Common vectors for introducing the therapeutic gene or nucleic acid include viral and non-viral vectors. Although viral delivery systems have been considered to be most efficient in delivering genes to cells, it may be limited because of a risk of triggering inflammatory or immunogenic responses. Forbes, S. J., Review Article: Gene Therapy in Gastroenterology and Hepatology, Aliment Pharmacol. Ther. 11:823-826 (1997).

Recently, the death of Jesse Gelsinger, a volunteer who died on Sep. 17, 1999 while participating in a gene therapy clinical trial at the Institute for Human Gene Therapy, University of Pennsylvania, has fueled the controversy over the use and safety of gene therapy. The trial was directed to treat ornithine transcarbmylase (OTC) using a modified adenoviral vector. The administration, however, of the vector to Gelsinger “initiated an unusual and deadly immune-system response that led to multiple organ failure and death.” Preliminary Findings, The Institute of Human Gene Therapy, University of Pennsylvania Health System, Dec. 2, 1999, <http://www.med.upenn.edu/ihgt/findings.html>. Although adenoviral vectors offer several advantages over other viral vectors in that they can infect a wide range of cells and are not limited to replicating cells, as are retroviral vectors, adenoviral vectors may activate the immune system, as seen in the Gelsinger's case, such that the initial dose or repeated introduction may become less effective, if not life threatening. See also Forbes, S. J., supra.

Moreover, the potential immune response to gene therapy is not limited to the vector used. Since the vector introduces a genetic sequence that encodes a protein, polypeptide, enzyme that may be seen as “foreign” to the host, immune responses toward the cells expressing that sequence and the products of that expression limit the effectiveness of the therapy. For example, in hemophilia experiments using the Factor VIII or IX gene as the therapeutic gene, antibodies generated against the newly expressed proteins may limit the effectiveness of the therapy. Forbes, S. J., supra; see also Herzog, R., Problems and Prospects in Gene Therapy for Hemophilia, Current Opinions in Hematology, 5:321-326 (1998). Cytotoxic T cells such as neutrophils may also attack these cells that expressed the “foreign” genes or viral vector genes, again limiting the effectiveness of gene therapy over a sustained period of time.

Thus, there exists a need to enhance the safety and efficiency of gene therapy by alleviating some of the detrimental effects of the immune system.

### SUMMARY OF THE INVENTION

The present invention involves the use of alpha-MSH and/or its derivatives as an adjunct to gene therapy. In one aspect of the invention, the gene therapy vector includes nucleic-acid sequences that express alpha-MSH and/or its derivatives, and inflammatory or immune-response gene promoters may control their expression. The sequences may also be expressed together with a therapeutic gene using an internal ribosomal entry site sequence. In another aspect of the invention, pharmacologically effective amount of alpha-MSH and/or its derivatives may be administered before, after, and/or concurrently with the gene therapy vector carrying the appropriate gene.

### GENERAL DESCRIPTION OF THE INVENTION

The references cited below are hereby incorporated by reference as if fully set forth herein. The present invention involves a method and system for gene therapy using alpha-melanocyte stimulating hormone (“α-MSH”) and/or its derivatives as an adjunct therapy. Because of its anti-inflammatory and anti-pyretic activities, (X-MSH and/or its derivatives may supplement gene therapy applications by limiting the inflammatory response of the patients to the gene therapy vector or the expressed protein. Unlike other immunosuppressants, α-MSH and/or its derivatives also possess antimicrobial properties that may simultaneously protect the body against infection and limit the negative effects of the immune system.

In one aspect of the invention, a pharmacologically effective amount of α-MSH and/or its derivatives may be administered before or after the gene therapy vector carrying the appropriate therapeutic gene or nucleic acid is administered. Alternatively, α-MSH and/or its derivatives may also be administered together with the gene therapy vector as a cocktail. The gene therapy vector may include both viral vectors such as adenoviral, retroviral, lentiviral, or adenovirus-associated viral (AAV) vectors, and non-viral vectors such as liposomes, calcium phosphate, antibodies or receptor based transfer vectors, electroporation, or direct injection of nucleic acids.

In another aspect of the invention, the gene therapy vector carries a DNA molecule that includes sequences for expressing α-MSH and/or its derivatives in the host cells. Using conventional molecular cloning techniques, gene sequences for α-MSH and/or its derivatives may be cloned into viral vectors or expression plasmid vectors. Constitutive promoters such as cytomegalovinis (CMV) promoter or inducible promoters may drive their expression. Preferably, the inducible promoter employs the use of inflammatory gene promoters such as the interleukins, in particular, the IL-6 promoter, or the complement system gene promoters. Other inflammatory gene promoters include promoters for TNF-α or the NF-εB response element.

The gene therapy vector carrying the α-MSH and/or its derivatives may also carry the appropriate therapeutic gene or nucleic acid of interest. This localizes the expression of α-MSH and/or its derivatives to the area expressing the therapeutic gene of interest. The local effect of α-MSH and/or its derivatives may inhibit a local inflammatory response without compromising the systemic immune system of the host. It may also protect the cells that have incorporated the gene therapy vectors from the inflammatory cytotoxic killings of neutrophils or T-cells. In a preferred embodiment of the invention, cloning the gene for α-MSH and/or its derivatives and associated promoter in the same DNA vector carrying the therapeutic gene of interest may achieve this co-localization effect.

Alternatively, the a-MSH and/or its derivatives can be expressed with an internal ribosomal entry site (IRES). The IRES sequence may be placed between the therapeutic gene and the gene for α-MSH and/or its derivatives. Thus, the two genes may be transcribed as a bicistronic mRNA transcript from a single promoter, and the bicistronic mRNA, in turn, may be translated simultaneously at the 5′ end and at the IRES sequence. Because both the protein from the therapeutic gene and α-MSH and/or its derivatives are produced from a single transcript, it is more likely that a single cell will express both proteins. IRES sequences and vectors can be commercially obtained, for example, from Clontech Laboratories, Palo Alto, Calif. (PIRES, cat# 6028-1).

Furthermore, stringing multiple genes for α-MSH and/or its derivatives using multiple IRES sequences may increase the production of α-MSH and/or its derivatives. A secretion signal peptide cloned upstream of the gene for α-MSH and/or its derivatives may also transport α-MSH and/or its derivatives to the extracellular environment where they are needed. Examples of such secretion peptide signal include the signal peptides for epidermal growth factor, basic fibroblast growth factors, or interleukin-6.

### EXAMPLE I

This example illustrates the anti-inflammatory, anti-pyretic, and anti-microbial activities of α-MSH and its derivatives.

α-MSH is an ancient thirteen amino-acid peptide, SYSMEHFRWGKPV (α-MSH (1-13)), that is produced by post-translational processing of the larger precursor molecule propiomelanocortin. It shares the 1-13 amino acid sequence with adrenocorticotropic hormone (“ACTH”), also derived from propiomelanocortin. α-MSH is known to be secreted by many cell types including pituitary cells, monocytes, melanocytes, and keratinocytes. It can be found in the skin of rats, in the human epidermis, or in the mucosal barrier of the gastrointestinal tract in intact and hypophysectomized rats. See e.g. Eberie, A. N., The Melanotrophins, Karger, Basel, Switzerland (1998); Lipton, J. M., et. al., Anti-inflammatory Influence of the Neuroimmunomodulator α-MSH, Immunol. Today 18, 140-145 (1997); Thody, A. J., et.al., MSH Peptides are Present in Mammalian Skin, Peptides 4, 813-815 (1983); Fox, J. A., et.al., Immunoreactive α-Melanocyte Stimulating Hormone, Its Distribution in the Gastrointestinal Tract of Intact and Hypophysectomized Rats, Life. Sci. 18, 2127-2132 (1981).

α-MSH and its derivatives are known to have potent antipyretic and anti-inflammatory properties, yet they have extremely low toxicity. They can reduce production of host cells' proinflammatory mediators in vitro, and can also reduce production of local and systemic reactions in animal models for inflammation. The “core” α-MSH sequence (4-10), for example, has learning and memory behavioral effects but little antipyretic and anti-inflammatory activity. In contrast, the active message sequence for the antipyretic and anti-inflammatory activities resides in the C-terminal amino-acid sequence of α-MSH, that is, lysine-proline-valine (“Lys-Pro-Val” or “KPV”). This tripeptide has activities in vitro and in vivo that parallel those of the parent molecule. The anti-inflammatory activity of o-MSH and/or its derivatives are disclosed in the following two patents and references, which are hereby incorporated by reference as if fully set forth the herein: U.S. Pat. No. 5,028,592, issued on Jul. 2, 1991 to Lipton, J. M., entitled Antipyretic and Anti-inflammatory Lys Pro Val Compositions and Method of Use; U.S. Pat. No. 5,157,023, issued on Oct. 20, 1992 to Lipton, J. M., entitled Antipreytic and Anti-inflammatory Lys Pro Val Compositions and Method of Use; see also Catania, A., et. al., α-Melanocyte Stimulating Hormone in the Modulation of Host Reactions, Endocr. Rev. 14, 564-576 (1993); Lipton, J. M., et.al., Anti-inflammatory Influence of the Neuroimmunomodulator of -MSH, Immunol. Today 18, 140-145 (1997); Rajora, N., et. al., α-MSH Production Receptors and Influence on Neopterin, in a Human Monocyte/macrophage Cell Line, J. Leukoc. Biol. 59, 245-253 (1996); Star, R. A., et. al., Evidence of Autocrine Modulation of Macrophage Nitric Oxide Synthase by α-MSH, Proc. Nat'l. Acad. Sci. (USA) 92, 8015-8020 (1995); Lipton, J. M., et.al., Anti-inflammatory Effects of the Neuropentide α-MSH in Acute Chronic and Systemic inflammation, Ann. N.Y. Acad. Sci. 741, 137-148 (1994); Fajora, N., et.al., α-MSH Modulates Local and Circulating tumor Necrosis Factor-α in Experimental Brain Inflammation, J. Neurosci, 17, 2181-2186 (1995); Richards, D. B., et. al., Effect of α-MSH (11-13) (lysine-proline-valine) on Fever in the Rabbit, Peptides 5, 815-817 (1984); Hiltz, M. E., et. al., Anti-inflammatory Activity of a COOH-terminal Fragment of the Neuropentide α-MSH, FASEB J. 3, 2282-2284 (1989).

α-MSH derivatives include, but are not limited to, peptides with the amino-acid sequence KPV (α-MSH (11-13)), MEHFRWG (α-MSH (4-10)), or HFRWGKPV (α-MSH (6-13)). These derivatives may also include homodimers or heterodimers of the above peptides, which may be achieved by adding cysteine residues at the N terminal of any of the above polypeptides and allowing the cysteines of two polypeptides to form a disulfide bond. The peptides may also be N-acetylated and/or C-aridated.

Substituting or deleting certain amino acid residues may also create biologically functional derivatives without altering the effectiveness of the peptides. For example, it is known that stabilization of the α-MSH sequence can greatly increase the activity of the peptide and that substitution of D-amino acid forms for L-forms can improve or decrease the effectiveness of the peptides. A stable analog of α-MSH, [Nle⁴,D-Phe⁷]-α-MSH, which is known to have marked biological activity on melanocytes and melanoma cells, is approximately ten times more potent than the parent peptide in reducing fever. Further, adding amino acids to the C-terminal of an α-MSH(11-13) sequence can reduce or enhance antipyretic potency. Addition of glycine to form the 10-13 sequence slightly decreased potency; the 9-13 sequence was almost devoid of activity, whereas the potency of the 8-13 sequence was greater than that of the 11-13 sequence. It is known that Ac-[D-K11]-α-MSH 11-13-NH2 has the same general potency as the L-form of the tripeptide (α-MSH (11-13). However, substitution with D-proline in position 12 of the tripeptide rendered it inactive, see e.g. Holdeman, M., et. al., Antipyretic Activity of a Potent α-MSH Analog, Peptides 6, 273-5 (1985). Deeter, L. B., et. al., Antipyretic Properties of Centrally Administered α-MSH Fragments in the Rabbit Peptides 9, 1285-8 (1989). Hiltz, M. E., Anti-inflammatory Activity of α-MSH (11-13) Analogs: Influences of Alterations in Stereochemistry, Peptides 12, 767-71 (1991).

Biological functional equivalents can also be obtained by substitution of amino acids having similar hydropathic values. Thus, for example, isoleucine and leucine, which have a hydropathic index +4.5 and +3.8, respectively, can be substituted for valine, which has a hydropathic index of +4.2, and a protein having like biological activity can still be obtained. Alternatively, at the other end of the scale, lysine (−3.9) can be substituted for arginine (−4.5), and so on. In general, it is believed that amino acids can be successfully substituted where such amino acid has a hydropathic score of within about +/−1 hydropathic index unit of the replaced amino acid.

As to the anti-microbial properties of α-MSH and/or its derivatives, they have been described in PCT Application Serial No. PCT/US00/06917, published as WO00/59527, entitled “Anti-microbial Amino Acid Sequences. Derived from alpha-Melanoctye Stimulating Hormone, filed Mar. 17, 2000 by inventors Catania, A. and Lipton, J. claiming priority to U.S. Provisional Patent Application Serial No. 60/126,233 entitled Antimicrobial Amino-Acid Sequences Derived from Alpha-Melanocyte Stimulating Hormone, filed Mar. 24, 1999. The above PCT publication and the provisional patent application are hereby incorporated by reference as if fully set forth herein.

### EXAMPLE II

This example illustrates the use of α-MSH and/or its derivatives in conjunction with gene therapy.

Preparation and purification of α-MSH and/or its derivatives may employ conventional solid-phase peptide synthesis and reversed-phased high-performance liquid-chromatography techniques. Patients who will undergo gene therapy may receive a pharmacologically effective amount of α-MSH and/or its derivatives either through injections or oral administration. The injections, for example, can be performed intravenously, intraperitionally, or intradermally depending on the specific location targeted by the gene therapy. After administration of α-MSH and/or its derivatives and under supervision of a physician, the patient can then receive a pharmacologically effective amount of the gene therapy vector containing the therapeutic gene or nucleic acid of interest according to conventional gene therapy protocols. If needed, additional administration of α-MSH and/or its derivatives may be given following the administration of the gene therapy vector.

Alternatively, the delivery cocktail for the gene therapy vector may include a pharmacologically effective amount of α-MSH and/or its derivatives that is concurrently or simultaneously administered to the patients.

### EXAMPLE III

This example illustrates the construction of gene therapy vector that expresses α-MSH and/or its derivatives.

Preparation and purification of gene sequences that express α-MSH and/or its derivatives may use, among other techniques, conventional oligonucleotide synthesis techniques. Complementary oligonucleotides can be made and annealed to form double stranded DNA molecules capable of being cloned. Additional sequences representing appropriate restriction enzyme sites may be engineered at the ends of each oligonucleotide. Preferably, the oligonucleotide sequence downstream of the α-MSH sequences includes a stop codon (TAG).

In addition, using polymerase chain reaction, a fragment corresponding to the signal peptide of IL-6 cDNA, nucleotides 33 to 120 (Genbank Accession No. J03783), may be synthesized, cloned into a vector such as pBluescript KS (Stratagene, San Diego, Calif.). Similarly, promoter regions for IL-6 or NF-εB may also be synthesized Using oligonucleotides with appropriate matching restriction enzyme sites and cloned upstream of the pBluescript carrying signal sequence. Using standard restriction enzyme digestion and DNA ligation, α-MSH or its derivatives sequences may be ligated to the signal sequence and the promoter.

If an internal ribosomal entry site (IRES) sequence is desired, the oligonucleotides sequence above may include such a sequence or it can be incorporated into PCR primers and linked by conventional PCR techniques. Alternatively, the α-MSH and/or its derivatives may be cloned into the pIRES vector from Clontech Laboratories. Multiple α-MSH and/or its derivatives may be constructed with multiple IRES sequence if so desired. An effective amount of the expression plasmid containing these constructs and the therapeutic gene of interest can be directly injected or introduced into patients using non-viral vectors such as liposomes, electroporation, or using a gene gun.

Alternatively, the α-MSH and/or its derivatives constructs can be inserted into appropriate replication deficient retroviral, lentiviral, adenoviral, or adenovirus-associated-viral vectors using standard restriction enzyme and ligation techniques, blunt end cloning, or PCR techniques. Packaging cell lines using helper viruses may then package the vector DNA into viral particles for use in gene therapy.

Titer of the recombinant virus may first be determined, and appropriate amount of viral particles may be introduced into the patients or hosts. It is understood that the viral vector may already contain a therapeutic gene or nucleic acid in addition to α-MSH and/or its derivatives.

Once the recombinant virus is introduced into cells, the cells may express α-MSH and/or its derivatives, which in turn, inhibits inflammation. The anti-inflammatory effect of α-MSH expression in cells through inhibition of NF-εB activation have been reported in Ichiyama, et. al., Autocrine α-Melanocyte-Stimulating Hormone Inhibits NF-εB Activation in Human Glioma, J. Neurosci. Res. 58:684-689 (1999), and is hereby incorporated by reference as if fully set forth herein.

It is understood that modifying the examples above does not depart from the spirit of the invention. It is further understood that each example can be applied on its own or in combination with each other.

1. A gene therapy system comprising:
a gene therapy vector having at least one therapeutic gene or nucleic acid; and
a pharmacologically effective amount of α-MSH and/or its derivatives associated with the gene therapy vector.

2. The gene therapy system of claim 1 wherein the pharmacologically effective amount of α-MSH and/or its derivatives is administered before, after, or together with the gene therapy vector.

3. The gene therapy system of claim 1 wherein the pharmacologically effective amount of α-MSH and/or its derivatives is expressed from nucleic acids carried by the gene therapy vector.

4. The gene therapy system of claim 3 wherein the nucleic acid carried by the gene therapy vector includes a therapeutic gene or nucleic acid.

5. The gene therapy system of claim 3 wherein the pharmacologically effective amount of α-MSH and/or its derivatives is expressed under control of an inflammatory or immune response gene promoter.

6. The gene therapy system of claim 5 wherein the inflammatory gene promoter is the promoter for Interleukin-6.

7. The gene therapy system of claim 3 wherein the pharmacologically effective amount of α-MSH and/or its derivatives are expressed from a nucleic acid having at least one internal ribosomal entry site sequence.

8. The gene therapy system of claim 3 wherein the pharmacologically effective amount of α-MSH and/or its derivatives are expressed from a nucleic acid having at least one secretion signal peptide.

9. The gene therapy system of claim 1 wherein the gene therapy vector is a viral vector.

10. The gene therapy system of claim 9 wherein the viral vector is an adenoviral vector.

11. A method of gene therapy comprising:
administering a pharmacologically effective amount of α-MSH and/or its derivatives before, after, or concurrently with the administration of a gene therapy vector having at least one therapeutic gene or nucleic acid.

12. The method in claim 11 wherein the pharmacologically effective amount of α-MSH and/or its derivatives is expressed from nucleic acids carried by the gene therapy vector.

13. The method in claim 11 wherein the nucleic acid carried by the gene therapy vector includes a therapeutic gene or nucleic acid.

14. The method in claim 12 wherein the pharmacologically effective amount of α-MSH and/or its derivatives is expressed under control of an inflammatory or immune response gene promoter.

15. The method in claim 14 wherein the inflammatory gene promoter is the promoter for Interleukin-6.

16. The method in claim 12 wherein the pharmacologically effective amount of α-MSH and/or its derivatives is expressed from a nucleic acid having at least one internal ribosomal entry site sequence.

17. The method in claim 12 wherein the pharmacologically effective amount of α-MSH and/or its derivatives is expressed from a nucleic acid having at least one secretion signal peptide.

18. The method in claim 11 wherein the gene therapy vector is a viral vector.

19. The method in claim 11 wherein the viral vector is an adenoviral vector.
